(19) United States
(12) Patent Application Publication
(10) Pub. No.: US 2026/0144974 A1
(43) Pub. Date: May 28, 2026

(54) CRANIAL ACCESS PORT SYSTEM

(71) Applicant: Whisper Medical, Inc., Mountain View, CA (US)

(72) Inventors: Martin S. DIECK, Palo Alto, CA (US); Soren THOMPSON, New York, NY (US)

(73) Assignee: Whisper Medical, Inc., Mountain View, CA (US)

(21) Appl. No.: 19/455,133

(22) Filed: Jan. 21, 2026

Related U.S. Application Data
(63) Continuation of application No. PCT/US2024/039037, filed on Jul. 22, 2024.
(60) Provisional application No. 63/514,938, filed on Jul. 21, 2023.

(51) Int. Cl. A61M 39/02; A61M 39/00; A61M 39/22

(52) U.S. Cl. CPC A61M 39/0247; A61M 39/223; A61M 2039/0018; A61M 2039/025; A61M 2039/0261

(57) ABSTRACT

Apparatus and systems include an access port system comprising a access port, a conduit, a negative pressure source, at least one wing, and a flushing source. The access port can comprise a first end having one or more flanges, a second end having a threaded portion, a longitudinal axis, a tubular portion extending between the first end and the second end, and a first lumen and a second lumen extending through the tubular portion. The conduit can comprise a first end and a second end. The first end of the conduit can be configured for positioning over the one or more flanges on the first end. The negative pressure source can be configured for fluidly coupling to the first lumen. The negative pressure source can be configured to draw suction through the first lumen. The at least one wing can extend orthogonally outwardly from the tubular portion.

### RELATED APPLICATION INFORMATION

This application is a continuation of PCT Application Number PCT/US2024/039037 filed Jul. 22, 2024, which claims the benefit of U.S. Provisional Patent Application No. 63/514,938, filed on Jul. 21, 2023. The entirety of each of which is incorporated by reference.

### TECHNICAL FIELD

This application relates to systems and methods for removing and flushing fluid from the subdural region of a patient.

### INTRODUCTION

The subdural space of the human head is the space located between the brain and the lining of the brain, which is referred to as the dura mater or dura. Brain hemorrhages can cause subdural hematomas, causing blood to build up on the surface of the brain. Such hemorrhages can be caused by atrophy of the brain, head injuries, and linear deceleration of the brain, among other causes.

One form of treatment for subdural hematomas is a craniotomy operation, which entails the removal of a large portion of the skull, opening of the dura, and evacuation of the collection of blood. The craniotomy frequently necessitates the placement of a subdural drain, which comprises a tube extending through the hole created by the craniotomy and into the subdural space for removing any additional accumulation of blood or fluid. However, craniotomy procedures are highly invasive and generally carry significant risks to the patient and an extended recovery period. Further, a patient may need to undergo a burr hole operation, which comprises boring a hole in the skull to drain fluid from the brain area. Drains also do little to prevent re-accumulation of fluid in the subdural space, which frequently leads to the recurrence of subdural hematomas in the patient.

Cranial access ports can also be used in minimally invasive procedures to remove fluid accumulations from the brain. Current ports are generally made of stainless steel and can be manufactured via molding or machining. However, such ports can leave harmful metal artifacts in the patient and can have difficulties in manufacturing, especially limiting design opportunities for the port itself. Current ports may also have unwanted occlusions within the port itself due to challenges with flushing the port.

Therefore, there exists a need to provide a cranial access port according to the present invention that does not leave artifacts in the patient after use, provides more efficient and versatile design opportunities, and can provide for flushing of the brain and port.

### BRIEF SUMMARY OF THE INVENTION

The following summarizes some aspects of the present disclosure to provide a basic understanding of the discussed technology. This summary is not an extensive overview of all contemplated features of the disclosure and is intended neither to identify key or critical elements of all aspects of the disclosure nor to delineate the scope of any or all aspects of the disclosure. Its sole purpose is to present some concepts of one or more aspects of the disclosure in summary form as a prelude to the more detailed description that is presented later.

The present disclosure includes access ports and access port systems that include various components used with the access port. While the access ports described herein are discussed for use as cranial access ports as one example, the access ports can be used in any portion of the body. In one variation the access port system include a cranial access port for positioning in a hard tissue having a cranial access port, a conduit, a negative pressure source, at least one wing, and a flushing source. The cranial access port can comprise a first end having one or more uneven surfaces, a second end having a threaded portion, a longitudinal axis, a tubular portion extending between the first end and the second end, and a first lumen and a second lumen extending through the tubular portion. The conduit can comprise a first end and a second end. The first end of the conduit can be configured for positioning over the one or more flanges on the first end. The negative pressure source can be configured for fluidly coupling to the first lumen. The negative pressure source can be configured to draw suction through the first lumen. The at least one wing can extend orthogonally outwardly from the tubular portion and can comprise a concave surface. The concave surface can be positioned to allow pushing on the concave surface to rotate the cranial access port in a thread direction of the threaded portion when the cranial access port is positioned in the hard tissue. The flushing source can be configured for coupling to the lumen and can introduce fluid into the lumen.

The cranial access port can comprise one or more wings extending outwardly from the tubular portion. The one or more wings can each comprise a concave surface and a convex surface and can extend orthogonal to the longitudinal axis of the cranial access port such that rotation of the port in a first direction is led by the convex surface. The cranial access port can comprise flanges at the first end of the cranial access port. The tubular portion can comprise one or more threads at the second end of the cranial access port. The one or more threads can be configured for cutting. The tubular portion can comprise a groove extending longitudinally within.

The system can further comprise a clamp positioned around the conduit at the first end of the conduit. The clamp can be configured to provide a fluid tight seal between the conduit and the cranial access port. The clamp can comprise a ratchet. The tubular portion can comprise a side hub configured to couple to the negative pressure source. The first lumen can be formed by an inner catheter within the tubular portion. The inner catheter can be concentric with the tubular portion. The inner catheter can be coupled to an inner surface of the tubular portion via one or more bridges. The first lumen and the second lumen can be formed within the tubular portion. The first lumen and the second lumen can be circular. The first lumen and the second lumen can be D-shaped. The first lumen can have a greater area than the second lumen.

The second end of the tubular portion can comprise a one-way valve within. The one-way valve can permit negative pressure towards the second end of the tubular portion. The system can further comprise a tool inserted through the lumen. The tool can be a cutting tool, a visualization tool, and/or a flushing catheter. The system can further comprise a three-way valve coupled to the second end of the cranial access port. The three-way valve can be configured to introduce suction, introduce fluid, and close the system such that fluid within the cranial access port is static. The lumen can taper outwardly from the second end to the first end. The system can further comprise a projection at the second end configured to direct fluid from the flushing source in a direction angled from the longitudinal axis. The system can further comprise a wall separating the first lumen and the second lumen. The wall can comprise gaps to direct fluid from the second lumen into the first lumen. The at least one wing can comprise a convex surface. The convex surface can lead rotation of the cranial access port in the thread direction.

In another variation, a cranial access port can be provided for positioning in a hard tissue and for use with a negative pressure source having a conduit and a flushing source. The cranial access port can comprise a first end having one or more flanges for receiving the conduit. The cranial access port can comprise a second end having a threaded portion for insertion into the hard tissue. The cranial access port can comprise a longitudinal axis extending between the first end and the second end. The cranial access port can comprise a tubular portion extending along the longitudinal axis. The cranial access port can comprise a first lumen and a second lumen extending through the tubular portion.

The cranial access port can comprise at least one wing extending orthogonally outwardly from the tubular portion. The at least one wing can comprise a concave surface positioned to allow pushing on the concave surface to rotate the cranial access port in a thread direction of the threaded portion when the cranial access port is positioned in the hard tissue. The negative pressure can be configured to draw a negative pressure through the conduit and the first lumen when the conduit is coupled to the first end. The flushing source can be configured to deliver fluid through the second lumen when the flushing source is coupled to the second lumen.

Other aspects, features, and embodiments of the present disclosure will become apparent to those of ordinary skill in the art upon reviewing the following description of specific example embodiments of the present disclosure in conjunction with the accompanying figures. While features of the present disclosure can be discussed relative to certain embodiments and figures below, all embodiments of the present disclosure can include one or more of the advantageous features discussed herein. In other words, while one or more embodiments can be discussed as having certain advantageous features, one or more of such features can also be used in accordance with the various embodiments of the disclosure discussed herein. In a similar fashion, while example embodiments can be discussed below as device, system, or method embodiments, it should be understood that such exemplary embodiments can be implemented in various devices, systems, and methods.

### BRIEF DESCRIPTION OF THE DRAWINGS

The present disclosure is best understood from the following detailed description when read with the accompanying figures.

FIG. 1A illustrates a perspective view of a cranial access port according to one variation of the invention.

FIG. 1B illustrates a top view of the cranial access port of FIG. 1A.

FIG. 1C illustrates a bottom view of the cranial access port of FIG. 1A.

FIG. 1D illustrates a front view of the cranial access port of FIG. 1A.

FIG. 1E illustrates a perspective view of a cranial access port according to another variation of the invention.

FIG. 2A illustrates a perspective view of a cranial access port according to another variation of the invention.

FIG. 2B illustrates a top view of a cranial access port according to one variation of the invention.

FIG. 3 illustrates a side view of another variation of the cranial access port system within a patient.

FIG. 4A illustrates a side view of another variation of the cranial access port having a side port.

FIGS. 4B-4E illustrate cross-sectional views of the proximal end of the cranial access port according to various variations of the invention.

FIG. 5 illustrates a side view of another variation of the cranial access port having a one-way valve and a tool therethrough.

FIG. 6 illustrates a side view of another variation of the cranial access port having a three-way valve.

FIG. 7A illustrates a cross-sectional view of the proximal end of the cranial access port according to another variation of the invention.

FIGS. 7B-7D illustrate side views of additional variations of the cranial access port having an irrigation wall therein.

### DETAILED DESCRIPTION

The detailed description set forth below, in connection with the appended drawings, is intended as a description of various configurations and is not intended to represent the only configurations in which the concepts described herein can be practiced. The detailed description includes specific details for the purpose of providing a thorough understanding of the various concepts. However, it will be apparent to those skilled in the art that these concepts can be practiced without these specific details. In some instances, well-known structures and components are shown in block diagram form in order to avoid obscuring such concepts.

All examples and illustrative references are non-limiting and should not be used to limit the claims to specific implementations and embodiments described herein and their equivalents. For simplicity, reference numbers can be repeated between various examples. This repetition is for clarity only and does not dictate a relationship between the respective embodiments. Finally, in view of this disclosure, particular features described in relation to one aspect or embodiment can be applied to other disclosed aspects or embodiments of the disclosure, even though not specifically shown in the drawings or described in the text.

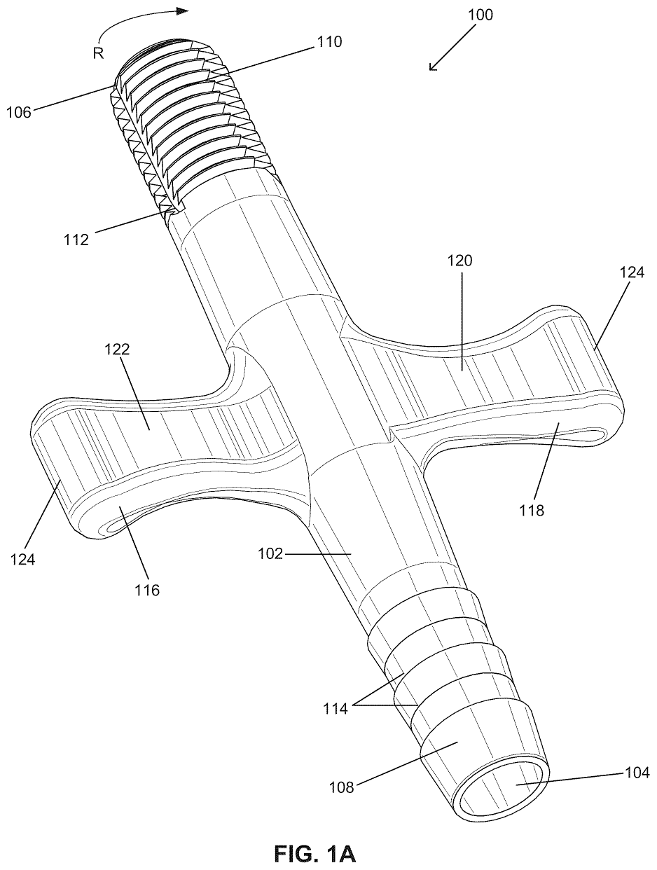

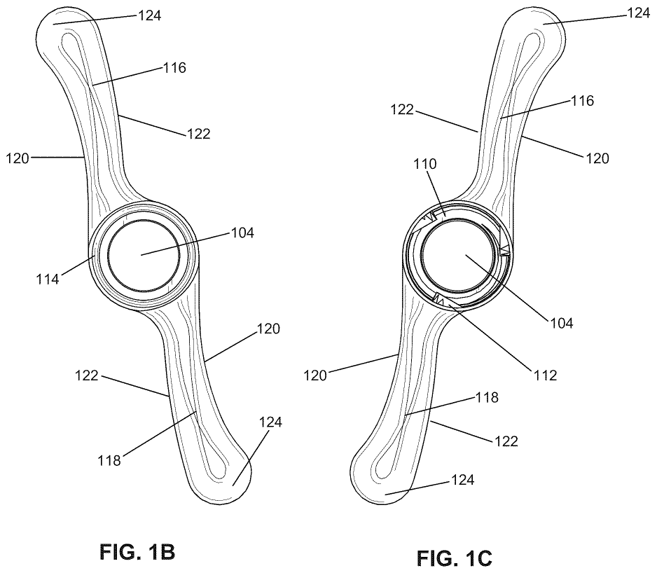

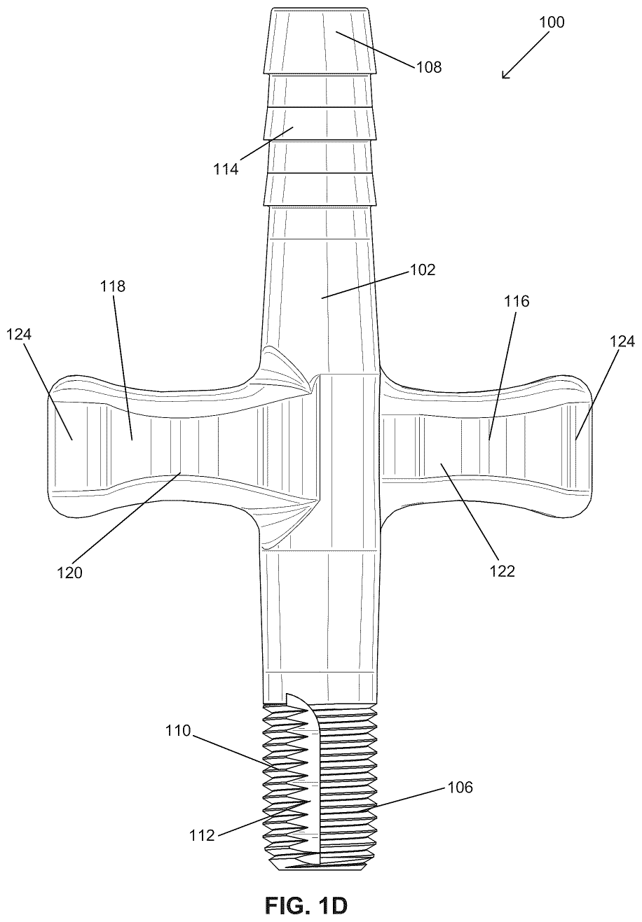

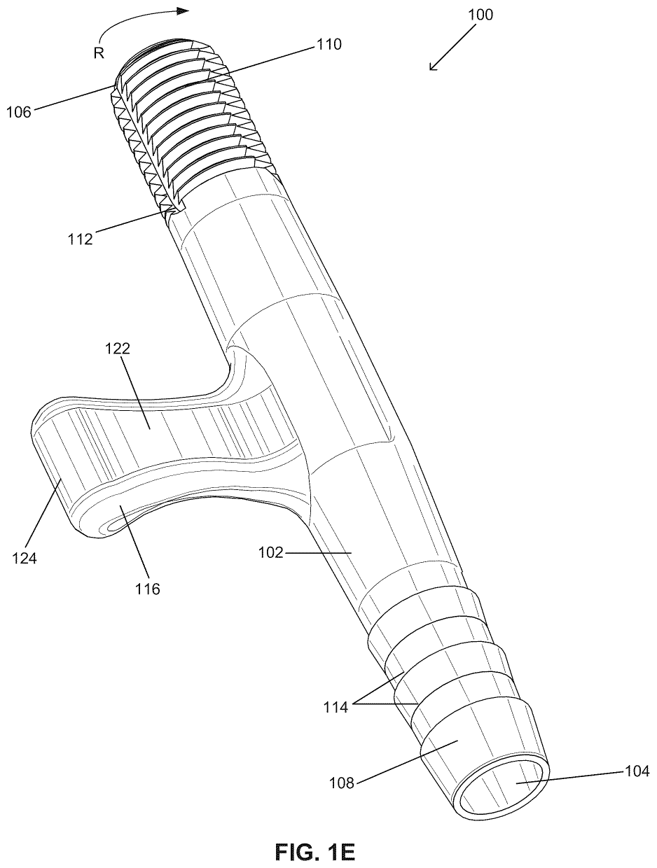

FIG. 1A illustrates a access port 100 comprising a tubular portion 102 extending along a length of the access port 100. The tubular portion 102 can have a lumen 104 that extends therethrough between a proximal end 106 and a distal end 108 of the access port 100. An exterior surface of the proximal end 106 of the tubular portion 102 can have one or more threads 110 formed thereon.

The access port 100 can be used for cranial procedures as well as other procedures in which a space is to be accessed via a port (e.g., arthroscopic procedures).

The one or more threads 110 can be used to cut into a skull of a patient as the proximal end 106 of the port 100 is inserted into a pre-formed opening in the patient's skull. The port 100 can be rotated within the opening to secure the port 100 therein. Additionally, a groove 112 may extend longitudinally within the threads 110 to create cutting surfaces within the threads 110, as best seen in FIG. 1D. The distal end 108 of the tubular portion 102 can have an uneven surface that serves to increase the ability of the port 100 to retain a conduit that is advanced over the distal end 108. In additional variations, this uneven surface can improve a fluid seal between the conduit 100 and the tubular portion. Examples of an uneven or irregular surface can include a roughened surface, a textured surface, one or more protrusions that are positioned on the surface, circumferential barbs, flanges, and recesses (e.g., that seats a clip or retainer positioned over the conduit). Alternatively, port 100 can be used in other locations of the anatomy such as other bones or hard tissues, soft tissues, connective tissues, or combinations thereof.

The access port 100 can includes wings 116, 118, extending outwardly from the tubular portion 102. Wings 116, 118 can facilitate rotation of the tubular portion into the opening of the skull during threading of the opening by the threads 110 of the access port 100. The wings 116, 118 can extend in opposite directions for enhancing the finger grip of the user of the wings 116, 118, and port 100. The wings 116, 118 can be mounted on the tubular portion at a location substantially medial between the proximal end 106 and the distal end 108 of the tubular portion 102, between the threads 110 and the uneven surface 114.

Wings 116, 118 can have concave surfaces 120 and convex surfaces 122 on opposite sides of the wings. Concave surfaces 120 can open substantially orthogonal to a longitudinal axis of the device such that a user's fingers can be placed within the surfaces 120 when rotating the port into the target area. When the access port 100 is positioned in the tissue, wings 116, 118 can allow pushing on the concave surface 120 to rotate the cranial access port in a thread direction of the threaded portion, as shown by arrow R. The thread direction can be either right-winding or left-winding with varying combinations of concave and convex surfaces on either side of the wings. In additional variations, the port can include standard wings without a concave/convex surface. For example, the concave surface can be replaced with a textured or other surface to assist in turning of the port. In other variations, the concave surface can be on an opposite surface to allow the users fingers to allow pushing on the concave surface when removing the port from tissue.

In some embodiments, wings 116, 118 can be removable from tubular portion 102. The wings 116, 118 can comprise a locking mechanism or post that can couple with a groove on the tubular portion. Accordingly, the wings 116, 118 can be removed as necessary to create a lower profile of the port 100 during use. In some embodiments, the wings 116, 118 can be made of a polymer.

As seen in FIGS. 1B and 1C, concave surfaces 120 and convex surfaces 122 can be offset from each other to provide ergonomic features for the user during use such that during deployment of the port 100, the convex surface 122 leads in front while the concave surface 120 is held by the user's fingers. Wings 116, 118 can also be provided with rounded bulges 124 at the ends of the wings 116, 118 to prevent slippage of the user's fingers during deployment. As seen in the variation in FIG. 1E, the access port 100 can have one wing 116.

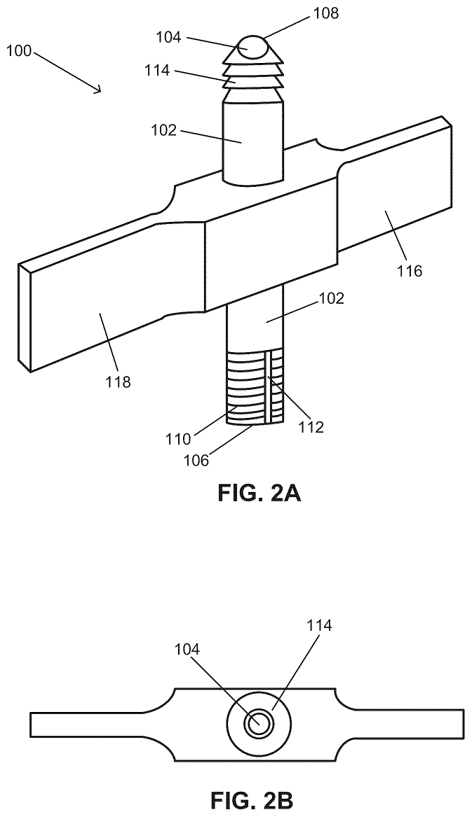

A radius between the convex surface 122 and the tubular portion 102 can be about 2.5 mm. A radius of the concave surface 120 can be about 17.5 mm. The overall radius of the device, when rotated, can be about 33 mm. As seen in the variation in FIGS. 2A and 2B, a access port 100 having flat wings 116, 118 can be provided.

In one embodiment of the port 100, an outer diameter of the exterior surface of the tubular portion 102 can be about 6.2 mm. The lumen 104 can have a diameter of about 4 mm at the distal end 108. The lumen can taper toward the proximal end 106 to a diameter of about 4.8 mm in order to achieve improved suction toward the proximal end. An overall length of the tubular portion 102 from the distal end 108 to the proximal end 106 can be about 50 mm. A width between tips of the wings 116, 118 can be about 23 mm. A width of the wings 116, 118 can be about 2.25 mm. The one or more threads 110 can extend about 10 mm from the proximal end, and the uneven surface 114 can extend about 15 mm from the distal end 108. In some variations, the threads 110 are configured to be self-tapping such that rotation of the threaded portion into tissue cuts or forms threads on the inside surface of the tissue.

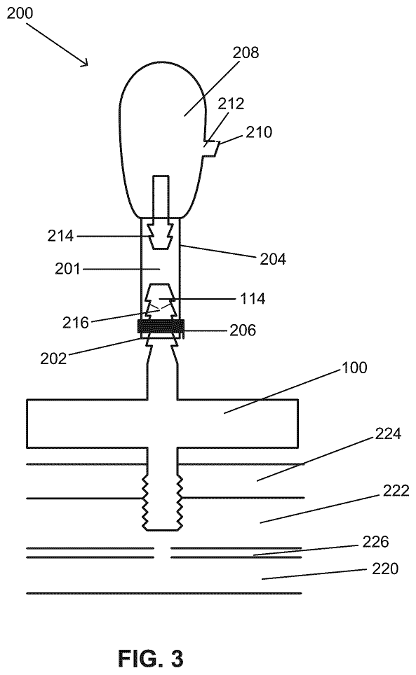

As seen in FIG. 3, another variation of the cranial access port system 200 can comprise a conduit 201 having a first end 202, and a second end 204, the second end coupled to a bulb 208. The conduit 201 can be coupled to the access port 100 at the first end 202. Uneven surface 114 can retain conduit 201 to the access port 100 to create a tight seal around the port 100 and the lumen 104. Optionally, a conduit clamp 206 can be placed around the conduit 201 to tighten the seal between conduit 201 and the port 100. The conduit clamp 206 can have a ratchet mechanism to tighten the clamp 206 as necessary. Alternatively, a suture can be tied around conduit 201 to seal the conduit 201 around the port 100.

The second end 204 of the conduit 201 can be coupled to the bulb 208. The bulb 208 can have a bulb opening at which the second end 204 of the conduit 201 can be connected via one or more bulb flanges 214. To produce a negative pressure condition in the conduit 201 and the lumen 104 of the access port 100, the bulb 208 can be compressed with the cap 210 removed from the side opening 212 to expel air from the bulb 208 when desired.

The negative pressure condition created in the lumen 104 of the access port 100 can be fluidly coupled to the lumen 104 and can draw fluid collected in the subdural space through the lumen 104 and into the conduit 201, and into the interior of the bulb 208. Accordingly, the fluid collected in the interior of the bulb 208 can be periodically emptied from the bulb 208, and the negative pressure condition may be reapplied to the subdural space through the access port 100 using the bulb 208.

The negative pressure condition can be removed when drainage from the subdural space is no longer observed, or the desired re-expansion of the brain 220 in the subdural space has occurred, or as is determined to be medically advisable. To remove the access port 100, the device can be rotated (e.g., using the wings 116, 118) such that the threads 110 move the port 100 out of the opening within the skull 222.

In addition, the tubular portion 102 can have a one-way valve 216 for introducing a negative pressure source. The one-way valve 216 can provide suction when there is an excess of air backed within conduit 201. Pulling suction towards the distal end 108 of the port 100 can open the valve 216, while any fluid moving towards the proximal end 106 of the port 100 can close the valve 216. Alternatively, a pressure relief valve can be provided to the port 100 as a safety feature, for example, to ensure that not too much pressure is applied to the brain.

The method of the invention can permit evacuation of a collection of fluid from the subdural space within the skull 222 of a patient. The method can include determining the region of the scalp 224 of the patient that is adjacent to the location of the collection of fluid in the subdural space. The region can be located on the patient's scalp 224 where the collection of fluid has the greatest dimension or measurement in the subdural space of the skull 222. The location of the greatest dimension of the fluid collection can be determined by performing an imaging study of the head of the patient using computerized tomography or magnetic resonance imaging to determine the extent of the collection of fluid. Once the greatest dimension of the collection of fluid is determined, the location of the opening to be made through the skull to the subdural cavity can be selected on the scalp at a substantially central location corresponding to the greatest dimension of the collection of fluid.

The scalp 224 of the patient can be infiltrated with an anesthetic, such as by injecting the anesthetic into the scalp 224 in the region where the subdural collection of fluid has the greatest dimension. The anesthetic can be lidocaine or epinephrine, or other suitable anesthetic.

An incision can be created in the scalp 224 to expose the bone of the skull 222 of the patient. The incision can extend through the scalp 224, the subcutaneous tissue, the galea, and the periosteum. A retractor device can be introduced into the incision for holding the scalp 224 adjacent to the incision away from the operating area.

An opening can be created in the skull 222 of the patient using a boring or cutting tool, for example, a drill device. The size of the opening formed in the skull can be about 3 to 8 mm in diameter (e.g., 6 mm). The dura 226 can then be penetrated by incising the dura 226 of the patient using, for example, a unipolar cautery device. The underlying membranes can be transected with the unipolar cautery device. The opening can be sized to be used with a drill device that is standard in separate operating room kits.

The incision in the dura can provide a passage for removal of fluid that has collected in the subdural space. This removal can most preferably be performed through the use of the access port 100. The proximal end 106 of the access port 100 can be introduced into the opening in the skull 222. The access port 100 can be rotated in the opening such that the one or more threads 110 engage the sides of the opening and pull the proximal end 106 into the opening and secure the port device against unintentional withdrawal of the device from the opening. The dura 226 can be penetrated by the proximal end 106 of the access port 100 for placing the lumen 104 in fluid communication with the subdural area and any collection of fluid in the subdural space.

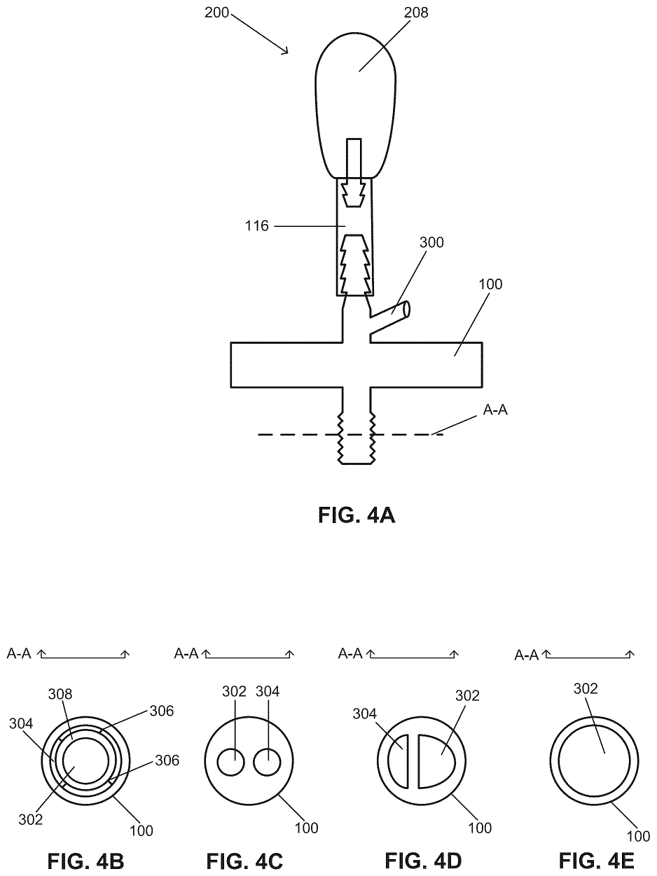

As seen in the variation of FIG. 4A, the tubular portion 102 of the port 100 can have a fluid port 300 the distal end 108 of the port 100. The fluid port 300 can be used for a connection to perform a “gentle flushing” for both the brain 220 and the access port 100 via a syringe (i.e., syringe 502) couplable to the fluid port 300. A saline solution can be delivered for the flushing. Flushing can be done in the event of occlusions in the port 100 and/or for clearance and subsequent removal of unwanted material (i.e., blood, film of excess material) or fluid within the brain, allowing the brain to heal more rapidly after an operation. The syringe can be limited to a certain amount of pressure via the fluid such that the user can control the fluid travelling towards the brain.

Ports that can both provide suction and adequate flushing can be beneficial for various reasons. For example, middle meningeal artery embolizations, which are embolizations in arteries in the brain that can create chronic subdural hematomas, can potentially be treated more commonly in patients. Accordingly, when middle meningeal artery embolizations are identified and/or treated, cranial access port evacuation can be used immediately afterwards, decreasing hospitalization time and risk of the subdural hematomas initially occurring. In such cases, one or more ports can be used to treat several hematomas as needed.

Cross section A-A at the proximal end 106 of the port 100 can show various lumen configurations that can be used within the port 100. For example, FIG. 4B shows a lumen configuration having an inner catheter 308 inserted through the lumen 104 of the port 100. A first lumen 302 and second lumen 304 can thus be created by the concentric inner catheter 308 within the port 100. The first lumen 302 can have a greater surface area and can be used for suction and the second lumen 304 can be used for flushing and irrigation, or vice versa.

The first and second lumens 302, 304 can be connected with bulb 208 or a syringe, respectively. The inner catheter 308 can be connected with an inner surface of the port 100 via extruded bridges 306 to provide structure such that the inner catheter 308 couples and is stable with respect to the port 100. The bridges 306 can be placed at various portions along the length of the port 100 such that fluid passing through the second lumen 304 can pass around the bridges 306.

In other variations, first lumen 302 and second lumen 304 can be used for various features, alone or in combination. Such methods can include suction, irrigation, tool insertion, and visualization.

Alternatively, the first lumen 302 and the second lumen 304 can be adjacent, as seen in FIGS. 4C and 4D. The first lumen 302 and the second lumen 304 can both be circular, D-shaped, or any combination thereof. Additionally, the configuration of FIG. 4D can provide for an increased area by which the suction line can utilize. As seen in FIG. 4E, access port 100 can have a single lumen through which both negative pressure and flushing can be performed.

The access port 100 can be manufactured via 3D printing for the purposes of manufacturing efficiency and flexibility in design opportunities. For example, 3D printing methods can enable the efficient manufacturing of designs such as those seen in FIGS. 4B to 3D. The port 100 can be made from titanium, stainless steel, PEEK (polyetheretherketone), polyetherimide, or ceramic, or a combination thereof. Using non-metallic materials can potentially reduce the risk of leaving metal and/or magnetic artifacts within the patient, which can cause negative effects beyond a subdural hematoma.

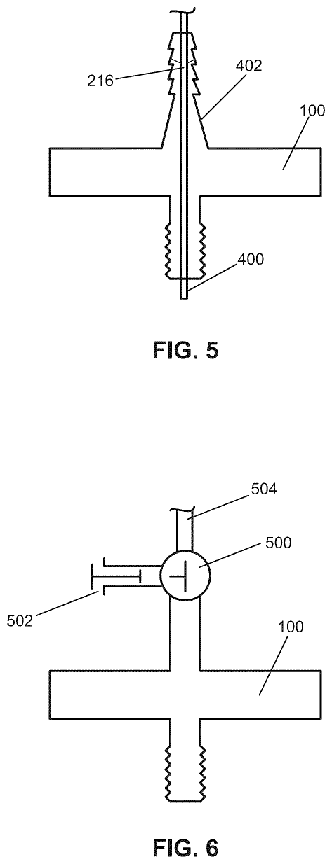

FIG. 5 shows a variation of the access port 100 having a tool 400, which can be introduced through lumen 104. A one-way valve 216 can be provided within the tubular portion 102 of the port 100 and can seal around the tool 400 when inserted through the port. The tool 400 can be a cutting device that can provide for injection of fluid therein. Alternatively, the tool 400 can be a cutting tool for making incisions within the dura 226. Alternatively, the tool 400 can be a visualization device that can provide visualization of pockets within the dura 226 that can be targeted for further suction or flushing. The visualization device can also provide illumination.

The tubular portion 102 of the port 100 can be provided with angled surfaces 402 for increased gripping surface for the user. Alternatively, surfaces 402 can be curved surfaces.

FIG. 6 shows a variation of the access port 100 having a three-way stopcock valve 500 at the distal end 108. The three-way valve 500 can be coupled to a syringe 502 for introducing a saline solution through the port 100 and to the brain 220. The three-way valve 500 can be screwed onto a hub on the port 100 to create an airtight and watertight seal. The three-way valve 500 can be connected to both a syringe 502 that can provide saline through the port 100 and a suction line 504 (connecting to bulb 208, for example) that can pull fluid through the port 100. The three-way valve 500 can also be turned off such that the system is static (i.e., nothing is pulled or pushed through the port 100).

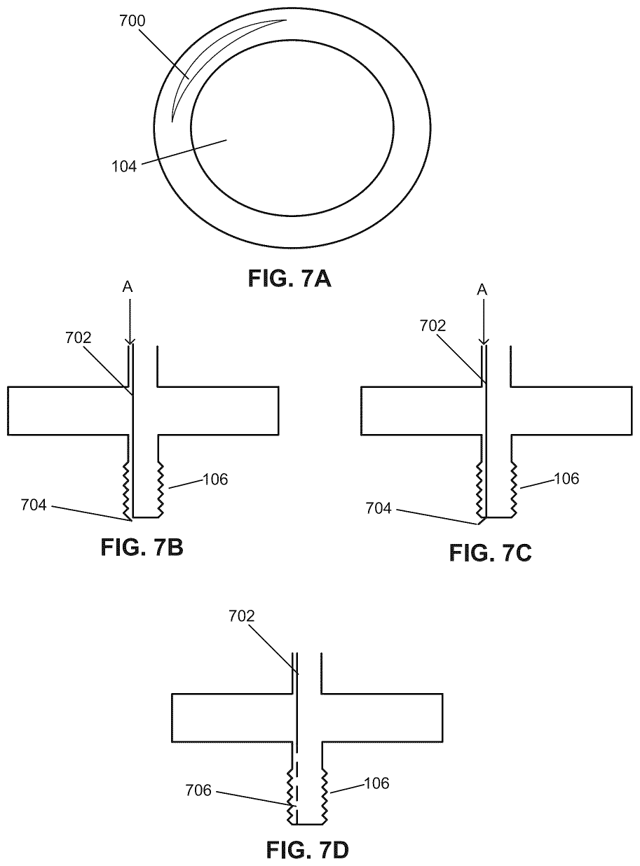

The cranial access port can also comprise various features for irrigation of fluid or debris within the target space. As seen in FIG. 7A, a flushing or irrigation lumen 700 can be provided within the wall of the tubular portion 102. Irrigation lumen 700 can have a crescent-like shape and can be used to rinse an area around the brain.

The tubular portion 102 can have an irrigation wall 702 within, which allows for fluid introduced into the irrigation lumen 700 to flow through the port 100 (see arrow A). As seen in FIGS. 7B and 7C, the distal end 108 of the port 100 can have a projection 704 or wing at the end, which can direct flow to the target area. The projection 704 can face towards or away from the longitudinal axis of the port 100 and can be adjustable by the user as necessary. Projection 704 can be shaped and/or sized to not damage the surrounding tissue when the port 100 is implanted. In the variation seen in FIG. 7D, irrigation wall 702 can have one or more gaps 706 to control and/or direct flow within the lumen. In this variation, flow may exit through lumen 104 of the port 100.

As used herein, including in the claims, “or” as used in a list of items (for example, a list of items prefaced by a phrase such as “at least one of” or “one or more of”) indicates an inclusive list such that, for example, a list of [at least one of A, B, or C] means A or B or C or AB or AC or BC or ABC (i.e., A and B and C).

As those of some skill in this art will by now appreciate and depending on the particular application at hand, many modifications, substitutions, and variations can be made in and to the materials, apparatus, configurations, and methods of use of the devices of the present disclosure without departing from the spirit and scope thereof. In light of this, the scope of the present disclosure should not be limited to that of the particular embodiments illustrated and described herein, as they are merely by way of some examples thereof, but rather, should be fully commensurate with that of the claims appended hereafter and their functional equivalents.

1. An access port system for positioning in a hard tissue, the system comprising:
an access port comprising a tubular portion having a first end and a second end, the first end having an uneven surface, a second end having a threaded portion, a longitudinal axis, and a first lumen extending through the tubular portion, wherein the first lumen tapers such that a first diameter of the lumen at the first end is greater than a second diameter of the lumen at the second end;
a conduit having a first end and a second end, wherein the first end of the conduit is configured for positioning over the uneven surface on the first end;
a negative pressure source configured for fluidly coupling to the tubular portion, wherein the negative pressure source is configured to draw suction through the first lumen;
at least one wing extending orthogonally outwardly from the tubular portion, wherein the at least one wing comprises a concave surface on a first side and a convex surface on a second side opposite to the first side, such the concave surface is positioned to allow pushing on the concave surface to rotate the access port in a thread direction of the threaded portion when the access port is positioned in the hard tissue; and
a flushing source configured for fluidly coupling to the tubular portion for delivery of a fluid through the tubular portion.

2. The access port system of claim 1, where the tubular portion further comprises a second lumen.

3. The system of claim 2, wherein the first lumen and the second lumen are circular.

4. The system of claim 2, wherein the first lumen and second lumen are D-shaped.

5. The system of claim 2, further comprising a wall separating the first lumen and the second lumen, wherein the wall comprises gaps to direct fluid from the second lumen into the first lumen.

6. The system of claim 1, wherein the negative pressure source comprises a bulb.

7. The system of claim 1, further comprising a three-way valve configured for coupling to the access port.

8. The system of claim 1, further comprising a clamp configured to be positioned around the conduit when attached to the first end.

9. The system of claim 1, wherein the tubular portion comprises a side port.

10. The system of claim 1, further comprising an inner catheter within the first lumen, wherein the inner catheter is concentric with the tubular portion.

11. The system of claim 10, wherein the inner catheter is coupled to an inner surface of the tubular portion via one or more bridges.

12. The system of claim 1, wherein the first lumen has a greater surface area than the second lumen.

13. The system of claim 1, wherein the second end of the tubular portion comprises a one-way valve, wherein the one-way valve permits negative pressure towards the second end of the tubular portion.

14. The system of claim 2, further comprising a cutting tool, a visualization device, or a flushing catheter advanceable through the second lumen.

15. The system of claim 1, further comprising a projection at the second end, wherein the projection is configured to direct fluid from the flushing source in a direction angled from the longitudinal axis.

16. The system of claim 1, wherein the at least one wing comprises a convex surface, opposite to the concave surface.

17. An access port system for positioning in a hard tissue, the system comprising:
an access port comprising a tubular portion having a first end and a second end, the first end having an uneven surface, a second end having a threaded portion, a longitudinal axis, and a first lumen and a second lumen extending through the tubular portion;
a conduit having a first end and a second end, wherein the first end of the conduit is configured for positioning over the uneven surface on the first end;
a negative pressure source configured for fluidly coupling to the tubular portion, wherein the negative pressure source is configured to draw suction through the first lumen; and
at least one wing extending orthogonally outwardly from the tubular portion, wherein the at least one wing comprises a concave surface on a first side and a convex surface on a second side opposite to the first side, such the concave surface is positioned to allow pushing on the concave surface to rotate the access port in a thread direction of the threaded portion when the cranial access port is positioned in the hard tissue; and
a flushing source configured for fluidly coupling to the tubular portion for delivery of a fluid through the tubular portion.

18. The access port system of claim 17, wherein the first lumen tapers such that a first diameter of the lumen at the first end is greater than a second diameter of the lumen at the second end.

19. An access port for positioning in a tissue and for use with a negative pressure source having a conduit and a flushing source, the access port comprising:
a tubular portion having a first end and a second end, the first end having an uneven surface configured to retain the conduit about the first end, the second end having a threaded portion and configured for insertion into the tissue;
a longitudinal axis extending between the first end and the second end;
a first lumen extending through the tubular portion, wherein the first lumen tapers such that a first diameter of the lumen at the first end is greater than a second diameter of the lumen at the second end; and
at least one wing extending orthogonally outwardly from the tubular portion, wherein the at least one wing comprises a concave surface positioned to allow pushing on the concave surface to rotate the access port in a thread direction of the threaded portion when the access port is positioned in the hard tissue.

20. The access port of claim 19, further comprising a second lumen extending through the tubular portion.

21. The access port of claim 19, wherein when the negative pressure draws a negative pressure through the conduit and the first lumen when the conduit is coupled to the first end.

22. The access port of claim 21, where the flushing source is configured to is configured to deliver a fluid through the second lumen.

23. The access port of claim 19, where the at least one wing is removable from the tubular portion.

24. The access port of claim 19, wherein the at least one wing comprises a convex surface, wherein the convex surface leads rotation of the access port in the thread direction.
